(19) United States
(12) Patent Application Publication
(10) Pub. No.: US 2007/0292360 A1
(43) Pub. Date: Dec. 20, 2007

(54) Process and composition for oral hygiene

(76) Inventors: Phyllis Siegel, San Antonio, TX (US); Bakul Bhatt, San Antonio, TX (US)

Correspondence Address: JOHN LEZDEY & ASSOCIATES, 2401 West Bay Drive, Suite 118, Largo, FL, 33770, US

(21) Appl. No.: 11/107,060

(22) Filed: Apr. 15, 2005

(51) Int. Cl. A61K 8/96; A61K 8/22

(52) U.S. Cl. 424/49; 424/50; 424/53

(57) ABSTRACT

A composition for oral hygiene which is anti-plaque and treats gingivitis. The composition contains a monobasic iodide salt to provide at least 30 ppm of available iodine. The composition includes an oxidizing agent, an organic acid and a buffer. There is also provided a method of treating gingivitis and the removal of biofilm.

### FIELD OF THE INVENTION

The present invention relates to a novel process and compositions for enhancing oral hygiene by removing and preventing plaque and calculus which comprises utilizing a mixture of a monobasic iodide salt, an organic acid and at least one oxidizing agent. The compositions are also effective in treating gingivitis and removal of biofilm without staining.

### BACKGROUND OF THE INVENTION

Prior oral hygiene formulations have been prepared with anti-plaque activity, but have not been able to avoid staining. Existing mouthwashes or oral rinses as well as tooth cleaning preparations can only minimize plaque formation and not reduce or eliminate existing plaque without staining. Cationic anti-plaque agents such as chlorhexidine and benzalkonium chloride are examples of the staining anti-plaque agents commonly used by dentists in treating gingivitis.

A variety of microorganisms are also present in the oral cavity. These range from the natural flora of the host to pathogenic species. Among these microorganisms are the gram-positive rods associated with the formation of plaque (a dense, enamel adherent, microorganism-containing polysaccharide matrix). Even with good oral hygiene, it has been shown that microorganisms (including those responsible for plaque formation) rapidly build up in the oral cavity. Specific areas, including periodontal and subgingival spaces, and interpapillary spaces of the tongue present environments that harbor bacteria. These spaces are difficult to reach by tooth brushing, and are only moderately affected by standard mouthwashes and dentifrices. The persistence of these microorganisms in such environments greatly increases, the risk of calculus and plaque build up and carie formation, which in turn presents the danger of gingival inflammation and periodontal disease.

Although mouthwashes are standard in oral hygiene, they have generally been used to mask halitosis. Several mouthwashes that have been marketed for the reduction of bacteria and the prevention of plaque buildup and gingivitis generally rely on a combination of alcohols (e.g. thymol, eucalyptol, ethanol; such as Listerine®), a combination of alcohols and a quaternary amine (e.g. ethanol, cetylpyridinium chloride; such as Scope®) or other oral surfactants (see U.S. Pat. No. 4,657,758), or of alcohol and chlorhexidine digluconate (Peridex® from Proctor and Gamble). However, the use of high amounts of alcohol in formulations tends to produce unpleasant side effects including pain and stinging of the oral mucosa, foul aftertaste and discoloration of teeth. Use of chlorohexidine results in stained teeth. Prior art attempts to address this issue have included the use of a cetylpyridinium chloride in the presence of an oral surfactant (Lander Alcohol Free Mouthwash from the Lander Company, Inc.) and the use of stabilized chlorine (RetarDent® from Rowpar).

### SUMMARY OF THE INVENTION

In accordance with the process of the invention, there is provided anti-staining anti-plaque compositions which are effective antimicrobials and are effective in inhibiting and reducing plaque in vivo without staining teeth and treating gingivitis. The Anti-staining, anti-plaque and anti-gingivitis compositions can be employed in mouthwash or oral rinse as well as in tooth cleaning preparations.

The present invention provides an anti-plaque composition for removing plaque and treating gingivitis comprising an aqueous solution containing an anti-plaque and anti-gingiviral effective amount of a monobasic iodide salt, an effective amount of an organic acid having up to eight carbon atoms, an effective amount of at least one oxidizing agent and a buffer, preferably a phosphate buffer, which can be incorporated in mouth washes or tooth cleaning preparations. More particularly, present composition comprises a monobasic iodide salt which is an alkali metal salt, preferably sodium, calcium, magnesium or potassium iodide in an amount of at least about 0.01 to 0.5% by weight, more preferably about 0.01 to 0.1%, an organic acid having up to eight carbon atoms, preferably selected from the group consisting of citric acid, ascorbic acid, and oxalic acid or the salts thereof in an amount of about 0.1 to 1% by weight, preferably 0.1 to 0.5%, an oxidizing agent and a buffer.

The oxidizing agent is preferably the alkali metal salt of a per acid or urea hydrogen peroxide which is present in an amount of at least about 0.01 to 1.0% by weight or other oxidizing agents used in whitening of teeth. There is an available iodine of at least 30 ppm, preferably about 80 to 250 ppm.

The composition is buffered to a pH of 2.3 to 6.0, preferably 3.0 to 3.5.

It is a general object of the invention to provide a non-staining anti-plaque and anti-gingivitis formulation for use in oral hygiene.

It is a further object of the invention to provide an anti-plaque composition which can be added to existing mouthwashes or teeth cleaning preparations.

It is yet another object to provide a composition which can remove or diminish existing plaque on teeth without tooth staining.

It is another object of the invention to provide a method for treating gingivitis.

It is still another object of the invention to reduce Candida biofilm.

### DESCRIPTION OF THE PREFERRED EMBODIMENTS

In accordance with the present invention there is provided an aqueous antimicrobial anti-plaque gingivitis treating composition that can be used alone or incorporated into a dentifrice or mouthwash which comprises:

A. an effective amount of a monobasic iodide salt to provide at least 30 ppm of available iodine to said composition, preferably about 80 to 250 ppm

B. an organic acid having up to eight carbon atoms;

C. an oxidizing agent, preferably an alkali salt of a per acid or urea hydrogen peroxide;

D. a buffer which can include the organic acid of part B, and;

E. water; said composition having an available iodine content of at least 30 ppm, preferably about 80 to 250 ppm.

The preferred anti-microbial anti-plaque composition which is added to the existing or prepared oral hygiene preparation comprises:

1) at least about 0.01 to 5% by weight of a monobasic iodide salt which is an alkali metal salt, preferably sodium or potassium iodide, so as to supply at least 80 to 250 ppm of available iodine;

2) about 0.01 to 2.0% by weight preferably about 0.01 to 2.0% by weight of an organic acid having up to eight carbon atoms, more particularly selected from the group consisting of citric acid, ascorbic acid and oxalic acid, or the alkali salts thereof,

3) about 0.009 to 1% by weight of an oxidizing agent selected from the group consisting of alkali salts of peroxide, sodium percarbonate, sodium perborate, sodium persulfate, urea hydrogen peroxide, peroxidase, ascorbic acid and mixtures thereof or other oxidizing agents used in the whitening of teeth having an oxidation potential greater than −0.54 electron volts, and

4) water, the composition can be buffered to a required pH to be used alone or alternatively after incorporated into a mouthwash or tooth cleaning preparation.

The anti-plaque composition is also anti-microbial so that it not only kills plaque forming pathogens but also removes the biofilm caused by the microbes. The composition is effective against a wide variety of various aerobic, anaerobic and facultative species, including Candida albicans, S. aureas, T. denticola, P. intermedia, cectinomyces, viscosus, P. gingivalis, S. sangrias, S. mutans, A. viscosus and A. naeslundii.

The composition can be used alone to directly treat plaque and/or gingivitis and to prevent its formulation or incorporated into conventional mouthwashes or tooth cleaning preparations provided that the iodine content of at least 30 ppm, preferably 100 to 250 ppm is maintained.

In general, the acid necessary to supply the required pH to the overall composition can be any organic or inorganic acid which does not chemically react with the other components, such as hydrochloric acid, phosphate salts, phosphoric acid, sulfuric acid, citric acid, acetic acid, preferably the organic acids or phosphate salts such as calcium pyrophosphate. The operating pH range for the composition is 2.3 to 4.0 and preferably, from about 2.8 to 3.3. The pH of an aqueous solution comprising the above enumerated components of the invention is determined by employing an aqueous solution of 0.5%, by weight, total of active components typically at a glass electrode, to precisely define the acidity of the composition.

In practice, the amounts of each of the components of the overall composition can range widely from 0.009 part to 40.0 parts by weight depending upon use. The balance after allowing for the acid is usually a physiologically acceptable solvent, such as water or a lower (C₁-C₄) monohydric aliphatic alcohol, for a total of 100 parts or more. Where water is employed, small amounts of a lower alkyl alcohol, such as ethanol or propanol, may be added thereto to provide easy formulation. The pH of the total composition is then adjusted to the requisite pH by adding a suitable inorganic or organic acid thereto. The defined components can be employed in mouth rinses, dental pastes, creams or tooth powders at concentrations ranging between 0.1 to 5.0% in a pH range from 2.3 to 4.0. Suitable non-reactive fluorides such as sodium fluoride or sodium monofluorophosphate also may be added. The critical percent is to maintain an available iodine content of at least 30 ppm, preferably 80 to 250 ppm. The lower pH has the greater amount of iodine parts per million.

When used against bacteria such as p. gingivalis or fungi the composition of the instant invention may be applied directly to the surface to be protected, or dissolved in a pharmaceutical carrier before application. Typically an effective amount, i.e., 0.025 to about 10% by composition is included in an inert carrier. Alternatively, an effective amount e.g., 0.025 to about 10% by weight may be incorporated into a solid carrier such as polishing agents and binding agents.

When compounds of the instant invention are prepared for oral use, they typically are incorporated in effective amounts up to about 10% by weight, preferably 0.05-3% by weight of the oral preparations. The oral preparation may be a liquid, such as a mouthwash. Mouthwash formulations typically contain water and 0-15% by weight of an aqueous lower aliphatic alcohol, such as ethanol, n-propyl alcohol or isopropyl alcohol. Preferably, the mouthwashes are non-alcoholic. Alternatively, the oral preparation may be a dentifrice, dental cream or powder. In such cases, an effective amount i.e., 0.025 to about 10% by weight may be incorporated into a solid, inert carrier, for example, selective polishing agents, and binding agents.

The dentifrice also may include humectants such as sorbitol, propylene glycol, gelling agents, (Irish moss) and sodium carboxy methyl cellulose, preservatives, silicones, chlorophyll compounds, flavoring or sweetening materials menthol and sucralose and compounds which provide fluorine containing ions such as sodium fluoride and sodium monofluorophosphate. Some classes of polishing agents, other than silica or equivalent types, adversely affect the anti-plaque properties attributable to antimicrobial agents used in cleaning teeth. Such polishing agents may be NaHCO₃, Al(OH)₃ or soluble sodium metaphosphate when incorporated in formulations show significant changes in pH after formulation. Silica-type polishing agents including, alumina calcined), aluminum silicate, zeolites, calcium pyrophosphate, dicalcium acid phosphate, kaolin and other inert polishing agents also are useful in this invention.

Thus, when the composition is a dentifrice containing a silica or equivalent type polishing agent, the dentifrice comprises a novel composition. As defined herein an inert polishing agent is one which is not reactive with the active ingredient surfactants of this disclosure nor with the protonating agents used for pH control and will not detract from the effectiveness of the dentifrice.

The oral preparations of the instant invention are typically applied to the oral cavity by brushing the teeth or rinsing the oral cavity at least with a mouthwash or irrigation device twice daily for about 10-90 seconds. Typical oral preparations of the invention which can be applied in this manner are set forth in the Examples described below.

To be effective as a mouthwash or irrigation formulation a composition must retain its stability and antimicrobial activity over the wide range of concentrations encountered at varied sites in the oral cavity. In addition, the efficacy of the active ingredient should not be diminished by any of the components of the vehicle in which it is contained. That is the composition should maintain an available iodine content of at least 30 ppm, preferably 80 to 250 ppm.

Attempts to use antimicrobial surfactants in the past to control plaque formation (and the resultant deposition of calculus leading to gingivitis and periodontal disease) have not proven successful. For example, the antimicrobial agents, chlorhexidine and benzalkonium chloride cause deposition of adherent stains on the hard surfaces of teeth and or dentures and prothese detracting from the possible utility of these agents in oral hygiene.

As indicated in U.S. Pat. Nos. 4,130,637 and 4,213,961 antimicrobial agents to be useful as aids in oral hygiene must inhibit plaque formation without staining. The anti-microbial agents of this disclosure are eminently suitable for oral hygiene preparations in that they control pathogens, help prevent plaque formation, do not cause objectionable staining.

The compositions of this disclosure show multiple modes of action. They include but are not necessarily limited to:

1. inhibition of glycolysis by plaque and caries causing microorganisms;

2. bacterial activity against oral pathogens such as P. gingivalis at low ppm concentrations of active ingredient; and

3. inhibition of bacterial adhesion to tooth surfaces (augmented in the presence of salivary proteins responsible for pellicle formation on the teeth).

It is believed that the latter unique characteristic is a likely reason for the sustained prevention of plaque adherence to tooth and prosthetic surfaces when composition is combined with selected polishing agents and used as a dentifrice.

The following examples illustrate the concentrated formulations of compositions useful in various oral hygiene preparations.

### EXAMPLE 1

A preferred 100 ml anti-plaque and anti-gingivitis composition of the present invention comprises;

| Ingredient | Wt. | |
|---|---|---|
| Sodium Iodide | 1.0-2.00 | g |
| Citric Acid | 0.01-1.08 | |
| Sodium Perborate | 0.01-0.05 | g |
| Sodium Carbonate | 0-0.05 | g |

| | |
|---|---|
| 1% Saline Solution | q.s. |

The salts may be dissolved in sterile water, saline solution or buffer solution with a pH of 2.3 to 4.0. For example, the preferred formulation for borax buffer is as follows:

Solution A-1 g Na₂P₄0₇ per 100 ml of H₂0.

Solution B-1.25 g H₃B0₃+0.3 g NaCl pr 100 ml H₂0 is mixed with Solution A and the salts are added. The mixture is then added to the anti-plaque or anti-gingivitis composition to obtain the desired pH.

The composition can be used by applying directly to the teeth or with a Water Pik®.

### EXAMPLE 2

A mouthwash was prepared by admixing the following
1.30 Kg of the mixture of Example 1
2.43 Kg Sucralose
2.28 Kg Glycerin USP
15.25 g. Saccharin USP
7.6 g. Sodium Fluoride USP
Citric Acid to adjust pH to 3.0 to 3.5

### EXAMPLE 3

A dentifrice formula is prepared by admixing the following

| | | |
|---|---|---|
| Carbowax 400 | 240 | g |
| Hydroxyethyl cellulose | 100 | g |
| Sodium saccharin | 214 | g |
| Menthol flavor | 0.2 | g |
| Calcium pyrophosphate | 2000 | g |
| Sodium iodide | 30 | g |
| Sodium perborate | 5 | g |
| Potassium Phosphate Monobasic | 5 | g |
| Citric acid | 10 | g |
| Water | 1000 | g |

The dentifrice is milled to a smooth paste.

### EXAMPLE 4

Commercially available teeth treatment compositions were tested against biofilm to determine efficacy in removal of biofilm by soaking in a solution.

| Product | Composition | Effect |
|---|---|---|
| 1. BIOVAC™ | 0.8 Chlorohexidine, | Slight |
| | 3.20% EDTA; | Stain |
| | Proteolytic enzymes | |
| | Dispersing agent | |
| 2. EFFERDENT™ | Potassium persulfate | Slight |
| | Sodium borate, | No Stain |
| | Sodium lauryl sulfate, | |
| | Sodium bicarbonate | |
| | Magnesium stearate | |
| 3. STERISOL | Chlorohexidine, | Slight |
| | Glycerol, 38F, | Stain |
| | Alcohol | |
| 4. Composition of | | Complete |
| Example 1 | | Removal |
| | | No Stains |

### EXAMPLE 5

An oral rinse was prepared by mixing the following ingredients:

| Ingredient | Wt. % |
|---|---|
| Citric Acid | 0.158 |
| Sodium Iodide | 0.0245 |
| Sodium Perborate | 0.033 |
| Potassium Phosphate Monobasic | 0.1 |
| Water | q.s. |

If desired, 0.1% Menthol and 0.03% Sucralose can be added in the above formulation and the quantity of water is adjusted accordingly.

### EXAMPLE 6

| Ingredient | Wt. % |
|---|---|
| Sodium Iodide | 0.0245 |
| Citric Acid | 0.157 |
| Sodium Persulfate | 0.0794 |
| Sodium Percarbonate | 0.033 |
| Water | q.s. |

### EXAMPLE 7

An oral rinse is prepared by admixing the following ingredients

| Ingredient | Wt. | |
|---|---|---|
| Potassium Mono or Dihydrogen Phosphate | 0-0.5 | g |
| Sodium Iodide | 0.01-0.06 | g |
| Citric Acid | 0.01-0.1 | g |
| Sodium Perborate | 0.009-0.41 | g |
| Menthol | 0.1% | by weight |
| Sucralose | 0.03% | by weight |

| | |
|---|---|
| 1% usp water | q.s. |
| The pH is adjusted to 3.3-3.8. | |

### EXAMPLE 8

### Object: To study the effect of Iocide formulations at three different pH levels on metabolic activity of cells in Candida albicans bio-film.

Methods: The three Iocides used in this experiment were: 1. Iocide pH 5.0, 80 ppm Iodine, 2. Iocide pH 6.4, 80 ppm Iodine and 3. Iocide pH 3.30, 150 ppm Iodine.

Candida Albicans bio-film was grown in three 96 wells micro-titer plates for 24 hours. The wells were carefully emptied and washed three times with Phosphate buffered saline to remove the unattached cells. In each plate, one row of eight wells was used as control. In other six rows of eight wells, the bio-film was exposed to 15 or 30 uL of each of the three Iocides listed above for one, five and twenty minutes. After the exposure time limit, the wells were carefully emptied and washed repeatedly with 100, 50 and 50 uL of PBS. A semi-quantitative measure of bio-film was calculated by using XTT reduction assay. Ramage, G. Et.al. (2001) Standardized method for in vitro antifungal susceptibility testing of Candida albicans biofilm. Antimicrobial Agents and Chemotherapy. 9:2475-2479. The percent inhibition of Candida albicans bio-film by these Iocide formulations was compared to the bio-film in the control wells.

### Results: The following table represents the summary of the percent inhibition of Candida bio-film by the three Iocide formulations at its ½ and full strengths and for the three exposure times.

| | Percent Inhibition | | |
|---|---|---|---|
| | Exposure Time: | | |
| | 1 minute | 5 minutes | 20 minutes |
| Cells + Iocide pH 5.0-30 uL | 48.00 | 63.89 | 66.96 |
| Cells + Iocide pH 6.4-30 uL | 78.57 | 93.33 | 95.21 |
| Cells + Iocide pH 3.3-30 uL | 84.44 | 95.74 | 94.84 |
| Cells + Iocide pH 5.0-15 uL | 38.89 | 29.47 | 50.28 |
| Cells + Iocide pH 6.4-15 uL | 50.44 | 54.77 | 64.77 |
| Cells + Iocide pH 3.3-15 uL | 58.22 | 53.12 | 50.42 |

The percent inhibition by the Iocide formulation pH 3.3 at full strength was 84.44, 95.74 and 94.84 after 1, 5, and 20 minutes exposures respectively and that by the Iocide formulation pH 6.4 was 78.57, 93.33 and 95.21 at the same exposure times. The percent inhibition was similar at 5 and 20 minute exposures by both of these formulations. The percent inhibition by these two Iocide formulations at ½ its strength was between 50 to 65% at all three exposure times. The percent inhibition of Candida bio-film by the Iocide formulation pH 5.0 was 48.00, 63.89 and 66.96 after 1, 5 and 20 minute exposure at full strength and 38.89, 29.47 and 50.28 at ½ its strength.

Conclusions: The Iocide formulation pH 3.3 was found to be more effective in inhibiting Candida albicans bio-film by 5 minutes exposure time. The effect of Iocide formulations pH 5.0 was less in inhibiting the Candida albicans bio-film.

What is claimed is:

1. A method for preventing and removing biofilm from teeth and treating gingivitis which comprises administering into the mouth of a patient a composition comprising:
A. an effective amount of a monobasic iodide salt to provide at least 30 ppm of available iodine to said composition;
B. an organic acid having up to eight carbon atoms;
C. an oxidizing agent,
D. a buffer, and
E. water,

2. The method of claim 1 wherein said organic acid is selected from the group consisting of citric acid, ascorbic acid oxalic acid or the alkali salts thereof, and said iodide salt is selected from the group consisting of sodium and potassium.

3. The method of claim 1 wherein said oxidizing agent is selected from the group consisting of the alkali salt of a peracid, peroxidase, alkali salts of peroxide and urea hydrogen peroxide.

4. The method of claim 3 wherein said oxidizing agent is selected from the group consisting of sodium percarbonate and sodium perborate.

5. The method of claim 1 wherein said buffer is a phosphate salt.

6. The method of claim 1 wherein said composition has a pH of 2.3 to 6.0.

7. The method of claim 1 wherein said composition is in a gel or a paste.

8. The method of claim 1 wherein the available iodine is about 80 to 250 ppm.

9. In an aqueous mouthwash for use in oral hygiene, the improvement which comprises that said mouthwash contains:
A. an effective amount of a monobasic iodide salt to provide at least 30 ppm of available iodine to said composition;
B. an organic acid having up to eight carbon atoms;
C. an oxidizing agent,
D. a buffer, and
E. water
said mouthwash has a pH of 2.3 to 6.0.

10. The mouthwash of claim 9 wherein said buffer is a phosphate salt.

11. The mouthwash of claim 9 wherein said organic acid is selected from the group consisting of ascorbic acid, citric acid, oxalic acid or the salts thereof.

12. The mouthwash of claim 9 wherein said monobasic salt is an alkali iodide.

13. The mouthwash of claim 12 wherein said alkali iodide is selected from the group consisting of magnesium iodide, calcium iodide, sodium iodide and potassium iodide.

14. In a dentifrice for preventing or removing biofilm or treating gingivitis, the improvement which comprises that said dentifrice contains:
A. an effective amount of a monobasic iodide salt to provide at least 30 ppm of available iodine to said composition;
B. an organic acid having up to eight carbon atoms;
C. an oxidizing agent, and
D. a buffer,

15. The dentifrice of claim 14 which is a gel.

16. The dentifrice of claim 14 which is a paste.

17. The dentifrice of claim 14 wherein said monobasic iodide salt is an alkali iodide.

18. The dentifrice of claim 17 wherein said alkali iodide is selected from the group consisting of sodium iodide and potassium iodide.
